(19) United States
(12) Patent Application Publication
(10) Pub. No.: US 2019/0191645 A1
(43) Pub. Date: Jun. 27, 2019

(54) METHOD FOR CULTIVATING PERENNIAL RICE USING ASEXUAL PROPAGATION CHARACTERISTIC OF ORYZA LONGISTAMINATA

(71) Applicants: YUNNAN UNIVERSITY, Yunnan (CN); FOOD CROPS RESEARCH INSTITUTE, YUNNAN ACADEMY OF AGRICULTURAL SCIENCES, Kunming (CN)

(72) Inventors: Fengyi HU, Yunnan (CN); Shilai ZHANG, Yunnan (CN); Liyu HUANG, Yunnan (CN); Dayun TAO, Yunnan (CN); Jing ZHANG, Yunnan (CN); Guangfu HUANG, Yunnan (CN); Jian HU, Yunnan (CN); Jinrong DAO, Yunnan (CN)

(21) Appl. No.: 16/327,465

(22) PCT Filed: Aug. 24, 2017
(86) PCT No.: PCT/CN2017/098803

§ 371 Date: Feb. 22, 2019

(30) Foreign Application Priority Data
Aug. 24, 2016 (CN) 201610717891.1

(51) Int. Cl. A01H 1/02

(52) U.S. Cl. CPC A01H 1/02

(57) ABSTRACT

A method for cultivating perennial rice having perenniality using an asexual propagation characteristic of Oryza longistaminata, wherein a perennial rice carrying a perennial genetic locus of Oryza longistaminata and adapting to different ecological types is cultivated, which makes it possible to harvest for two or more years by means of planting only once, thereby increasing efficiency of rice cultivation and production.

### FIELD OF THE INVENTION

The present invention belongs to the field of crop genetics and breeding, and particularly relates to a method for cultivating perennial rice by utilizing an asexual reproduction characteristic of Oryza longistaminata.

### BACKGROUND OF THE INVENTION

In response to problems such as soil erosion and declined soil fertility caused by long-term over-cultivation, the International Rice Research Institute (IRRI) proposed the development of perennial upland rice in 1989, and planting perennial upland rice on dry land, so as to achieve the dual purpose of preventing soil erosion and protecting the ecological environment while obtaining food income. Since then, some research institutions at home and abroad have begun to explore the genetic improvement of perennial rice. Perennial Rice (PR), as its name suggests, is a rice variety that can be harvested for many years, including its production techniques. Cultivated rice is commonly known as the modern improved rice variety, and whether it is Asian cultivated rice (O. saliva) or African cultivated rice (O. glaberrima), its wild species have perennial characteristics. In general, perennial rice is mainly obtained by using axillary buds on cultivated rice stems, stolons of common wild rice (O. rufipogon), and rhizomes of O. longistaminata to achieve the perennial nature of rice cultivation. However, axillary buds and stolons have not made more progress due to environmental factors such as low temperature and drought in winter, and the development of perennial rice using the characteristics of the rhizomes of Oryza longistaminata has gradually made new breakthroughs in theory and practice.

In 1991, the French scholar Ghesquiere studied that the expression of the underground stem of Oryza longistaminata and the embryo abortion gene (D1) are linked together on the chromosome 2 of rice. In 1998, Japanese scholar Maekawa studied that the rhizome (Rhz) of Oryza longistaminata was linked to the ligamentless gene (lg, liguleless) on chromosome 4. In 2003, Erik. J. Sacks of the International Rice Research Institute concluded that the expression of rhizome is controlled by recessive or partially recessive genes. However, the series of studies only stayed at the theoretical level, did not make substantial progress, and was not actually used for production.

### SUMMARY OF THE INVENTION

The present invention provides a method for cultivating perennial rice having perenniality using an asexual propagation characteristic of Oryza longistaminata, and realizes that the perennial rice plant can grow continuously for two or more years, and the present invention can also utilize the combination of different perennial genetic loci to cultivate perennial rice adapted to different ecological types, and improve the production efficiency of rice production.

The present invention is implemented by the following technical solutions:

A method for cultivating perennial rice using an asexual propagation characteristic of Oryza longistaminata, comprising the following steps:
(1) crossing Oryza longistaminata as the male parent and a first cultivated rice as the female parent to obtain a first F₁ generation;
(2) self-crossing the first F₁ generation to obtain a first F₂ generation;
(3) screening out a first F₂ generation carrying a perennial genetic locus, wherein the perennial genetic locus includes a major QTL locus and a minor QTL locus;
(4) continuously self-crossing the first F₂ generation carrying the perennial genetic locus to obtain a progeny material, from which a perennial rice line is selected and bred, and the perennial rice line carries the perennial genetic locus, that is, it has perennial trait.

In an aspect of the present invention, the method further comprises the steps of:
(5) introducing the perennial genetic locus into a second cultivated rice through hybridization to obtain a second F₁ generation using the perennial rice line with perennial trait selected by step (4) as a donor and the second cultivated rice as a female parent;
(6) backcrossing the second F₁ generation and the second cultivated rice as a male parent, and then self-crossing, and screening out the perennial rice carrying the perennial genetic locus.

In still another aspect of the invention, the first cultivated rice is RD23.

In still another aspect of the invention, the second cultivated rice is Chujing 28.

In still another aspect of the invention, the major QTL locus includes Rhz2 and Rhz3, and the minor QTL locus includes QRl1, QRbd12, QRn2, QRn3, QRn5, QRn6, QRl6, QRn7, QRl7 and QRl10.

In still another aspect of the invention, the perennial rice having perennial trait carries one or more perennial genetic loci selected from the group consisting of Rhz2, Rhz3, QRl1, QRbd2, QRn2, QRn3, QRn5, QRn6, QRl6, QRn7, QRl7 and QRl10.

In still another aspect of the invention, the progeny material obtained by continuous self-crossing of the F₂ generation includes one or more selected from the group consisting of F₃ generation, F₄ generation, F₅ generation, F₆ generation, F₇ generation, F₈ generation, F₉ generation, F₁₀ generation, F₁₁ generation and/or F₁₂ generation.

In still another aspect of the invention, the second F₁ generation is backcrossed four times with the second cultivated rice male parent, and then self-crossed for 4 generations.

The beneficial effects of the invention are:

1) The method for cultivating perennial rice provided by the present invention obtains progeny isolated population by hybridization of cultivated rice/Oryza longistaminata, and can accurately detect 12 genetic loci controlling perennial (rhizome expression and abundance); by using materials with different perennial genetic loci as donors, molecular marker-assisted selection (MAS) was used to breed perennial rice varieties (lines) that were planted once and could be harvested continuously for many years (multiple times); and the present invention can produce perennial water/upland rice adapted to different ecological types through the combination of different perennial genetic loci, such as perennial rice with long rhizomes suitable for dryland cultivation (such as PR12-1375); and perennial rice varieties with short rhizomes (such as PR24) suitable for common paddy field cultivation.

2) The perennial rice cultivated according to the method provided by the present invention has an essential difference in the regeneration mechanism compared with the ratoon rice. The perennial rice provided by the present invention is a re-growth of the axillary buds of the rhizomes controlled by the perennial genetic loci of Oryza longistaminata, and the rice production capacity returns to normal, and has the production advantage of harvesting for many years (multi-season) by planting once; compared with the existing ratoon rice technology, the perennial rice provided by the present invention has higher practical significance in perennial rice.

3) The perennial rice cultivated by the method provided by the present invention has high output for many years, has good economic benefits, and has great promotion value. The perennial rice production technology shows a new type of high-efficiency, environmentally friendly, green agriculture, light and simplified rice production and management. There is no need to repeat the purchase of seeds, field cultivation of seedlings, transplanting, and plowing fields, which greatly reduces labor and capital investment. According to statistics, compare to annual rice, the production cost of planting the perennial rice can be reduced by 45%, that is about 600 CNY/mu (Chinese acre, which equals to 666.67m², meanwhile, the yield, and the income is higher, resulting it extremely extension value.

### DETAILED DESCRIPTION OF THE INVENTION

The present invention will be further described in detail below in conjunction with the specific embodiments. It is to be understood that the description is not intended to limit the scope of the invention. In addition, descriptions of well-known structures and techniques are omitted in the following description in order to avoid unnecessarily obscuring the inventive concept.

In the present invention, Oryza longistaminata is used as a male parent, a wild species widely grown in tropical Africa, having long stigma and anthers (Oka, 1967), self-incompatibility (Nayar), 1968; Chu, 1969), cross-pollination (Causse, 1991) and rhizomes (Porteres, 1949; Bezancon, 1977; Ghesquiere, 1985). In Oryza longistaminata, due to the unique asexual reproduction of underground stems, it is an ideal trait for the development of perennial rice. The present invention is indeed based on the characteristic of Oryza longistaminata, thereby successfully cultivating perennial rice.

The cultivated rice used as a female parent in the present invention may be a majority of rice varieties including indica rice and japonica rice which are well known to those skilled in the art. In the present invention, the first cultivated rice and the second cultivated rice may be the same or different. In a preferred embodiment, the first cultivated rice is RD23, which is a widely grown indica variety from Thailand; and the second cultivated rice is Chujing 28, which is a japonica variety cultivated and widely cultivated from Chuxiong Agricultural Science Institute of Yunnan Province.

As used herein, the terms “perennial”, “perenniality” or similar terms refer to the line characteristics selected by the hybrid offspring of Oryza longistaminata and cultivated rice, which can be grown and harvested for many years (two or more years) by asexual reproduction, thereby having good perennial ability.

Using techniques well known to those skilled in the art, such as, but not limited to, hybridization, backcrossing and molecular marker assisted selection (MAS), Oryza longistaminata is used as the male parent, and the cultivated rice is used as the female parent to obtain the first F₁ generation. In an embodiment, the first F₁ generation is F₁ (RD23/O. longistaminata).

As used herein, the term “self-crossing” refers to the binding of a male or female gamete from the same individual or mating between individuals of the same genotype or between individuals from the same clonal breeding line.

As used herein, the term “backcrossing” refers to the hybridization of a child generation with any of the two parents, a method known as backcrossing. In breeding work, backcrossing methods are often used to enhance the performance of a parent in a hybrid individual. The offspring produced by the backcrossing method are called backcross hybrid. The parent who is used to backcross is called the recurrent parent, and the parent who is not used to backcross is called a non-recurrent parent.

In the present invention, the first F₂ generation is obtained by self-crossing the first F₁ generation. The present invention may also include obtaining a BC₁ segregating population by backcrossing the first F₁ generation (the recurrent parent is the female parent, and in a preferred embodiment is preferably the female parent RD23).

Genetic analysis was performed on the first F₂ generation and/or BC₁ segregating population to screen for perennial genetic loci. For the identification and analysis of perennial genetic loci in Oryza longistaminata, see Hu Fengyi's “Molecular Mapping and Genetic Study on Rhizome of Oryza longistaminata”, Master's Thesis of Southwest Agricultural University of Year 2002. This Thesis is incorporated herein in its entirety as a reference. Wherein the perennial genetic loci include a major QTL locus and a minor QTL locus, specifically:

In the present invention, the major QTL locus includes Rhz2 and Rhz3, wherein Rhz2 is located between the SSR molecular markers OSR16 and OSR13 on chromosome 3 with distances of 1.3 cM and 8.1 cM, respectively, and Rhz3 is located between the SSR molecular markers RM119 and RM237 on chromosome 4 with distances of 2.2 cM and 7.4 cM, respectively. It should be noted that the label names such as OSR16 are well known to those skilled in the art and belong to the rice SSR molecular marker term published based on the development of rice genome sequences.

As shown in Table 1, in the present invention, the minor QTL locus includes QRl1, QRbd2, QRn2, QRn3, QRn5, QRn6, QRl6, QRn7, QRl7 and QRl10.

As used in the present invention, Rhz represents the major QTL locus of the rhizome expression; Q represents the minor QTL locus (QTL); RN: the number of rhizomes per plant; RBD: degree of rhizome branching; RBN: secondary branching degree; RL: average length of rhizomes; RIL: average length of rhizome internodes; RIN: number of internodes; RDW: dry weight of rhizomes per plant; TN: number of tillers per plant; numbers indicate on which chromosome the locus is located.

TABLE 1
Rhizome related genes/QTLs and traits

| Loci | Chrome | Maker Interval | Trait | LOD | A | Pa | D | D/A |
|---|---|---|---|---|---|---|---|---|
| QRl1 | 1 | RM306-RM237 | RL | 3.1 | −1.17 | 0.0024 | 1.28 | −1.09 |
| | | | RIL | 3.4 | −0.16 | 0.0013 | 0.17 | −1.06 |
| Rhz2 | 3 | OSR13-OSR16 | RBD | 12.71 | −0.83 | 0 | | |
| | | | RIL | 10.91 | −0.53 | 0 | | |
| | | | TN | 6.64 | −21.7 | 0 | | |
| QRn3 | 3 | RM282-RM4551 | RN | 7.11 | −1.58 | 0 | | |
| | | | RBN | 8.51 | −0.51 | 0 | | |
| | | | RL | 7.78 | −3.15 | 0 | | |
| | | | RIN | 12.38 | −1.01 | 0 | | |
| Rhz3 | 4 | RM119-RM273 | RN | 3.85 | −0.6 | 0.0975 | 1.28 | −2.13 |
| | | | RBD | 14.39 | −0.89 | 0 | | |
| | | | RBN | 8.11 | −0.41 | 0.0001 | 0.34 | −0.83 |
| | | | RL | 13.12 | −1.93 | 0 | 2.31 | −1.2 |
| | | | RIL | 15.58 | −0.31 | 0 | 0.37 | −1.19 |
| | | | RIN | 7.46 | −0.66 | 0.0002 | 0.58 | −0.88 |
| | | | TN | 8.82 | −23.5 | 0 | 11.78 | −0.5 |
| | | | RDW | 2.31 | −0.25 | 0.4181 | 1.15 | −4.6 |
| QRn5 | 5 | RM161-RM421 | RN | 2.06 | −1.03 | 0.0022 | | |
| | | | RL | 2.85 | −1.22 | 0.003 | | |
| | | | RIL | 3.38 | −0.24 | 0.0001 | | |
| | | | RIN | 3.64 | −0.71 | 0.0001 | | |
| | | | RBD | 3.2 | −0.33 | 0.0017 | −0.25 | 0.76 |
| QRl6 | 6 | RM30-RM6309 | RBD | 2.92 | −0.48 | 0.0013 | | |
| | | | RL | 2.86 | −1.47 | 0.0004 | | |
| | | | RIL | 3.15 | −0.25 | 0.0002 | | |
| | | | TN | 4.13 | −17.3 | 0.0001 | | |
| QRl7 | 7 | RM336-RM234 | RBD | 2.77 | −0.31 | 0.0004 | | |
| | | | RL | 3.51 | −1.46 | 0.0002 | | |
| | | | RIL | 3.74 | −0.23 | 0.0001 | | |
| QRnl10 | 10 | RM271-RM269 | RN | 3.74 | −1.2 | 0.0001 | −0.65 | 0.54 |
| | | | RL | 4.34 | −1.59 | 0 | −0.8 | 0.5 |
| | | | RIL | 5.22 | −0.24 | 0 | −0.14 | 0.58 |
| | | | RIN | 3.81 | −0.61 | 0 | | |
| | | | RDW | 2.12 | −0.92 | 0.0035 | | |
| QRbd2 | 2 | RM71-RM300 | RBD | 4.21 | −0.39 | 0 | | |
| | | | RDW | 3.66 | −1.3 | 0.0001 | −0.75 | 0.58 |
| QRn6 | 6 | RM345-OSR21 | RN | 2.5 | −1.11 | 0.0018 | | |
| | | | RIN | 5.51 | −0.86 | 0 | −0.61 | 0.71 |
| QRn2 | 2 | RM341-RM327 | RN | 4.09 | −1.55 | 0 | −1 | 0.65 |
| QRn7 | 7 | RM125-RM180 | RN | 3.46 | −1.03 | 0.0006 | 0.75 | −0.73 |

In the present invention, the first F₂ generation carrying the perennial genetic locus is self-crossed to obtain one or more progeny materials from the group consisting of F₃ generation, F₄ generation, F₅ generation, F₆ generation, F₇ generation, F₈ generation, F₉ generation, F₁₀ generation, F₁₁ generation and/or F₁₂ generation, and the genome is substantially stable and homozygous to form a line, which can be used as the first perennial rice line carrying a perennial genetic locus. The first perennial rice line may include one or more rice lines according to the strain genotype (carrying different perennial genetic loci) and phenotype, and any perennial rice line may be selected for breeding according to needs. The first perennial rice line preferably used in the present invention is a rice line having perennial trait cultivated by molecular marker-assisted selection, such as the rice line of PR12-1375, PR23, PR24 and the like in the present invention, named according to Perennial Rice 12-1375, Perennial Rice 23, Perennial Rice 24. In a preferred embodiment, different lines carry different perennial genetic loci to obtain different types of perennial rice, such as perennial rice with long rhizomes, such as PR12-1375, which carries the perennial genetic loci Rhz2(Chr3), Rhz3(Chr4), QRn3(Chr3), QRbd2(Chr2), QRl7(Chr7), and QRn10(Chr10), is planted in the mountains to prevent soil erosion and protect ecology, while obtaining a certain amount of production; a short rhizome line, for example, PR24 carries the perennial genetic loci Rhz2(Chr3), Rhz3(Chr4), QRn2(Chr2), QRbd2(Chr2), QRn7(Chr7), QRn10(Chr10), which has very short rhizomes, can be perennial and have high yields.

In the present invention, the above first perennial rice line having different perennial genetic loci is used as a donor, and in a preferred embodiment, the PR23 and/or PR24 perennial rice line is used as a donor, and the perennial genetic locus was introduced into other cultivated rice by the hybrid method using the cultivated rice as the female parent. Perennial genetic improvement is carried out to obtain perennial rice with better traits.

The MAS technique is employed in the present invention, that is, the Molecular Marker-Assisted Selection (MAS), which utilizes molecular markers closely linked to the target trait gene for indirect selection, is the selection of the target trait at the DNA level, is not affected by the environment, is not interfered by the allelic recessive relationship, and the selection results are reliable, and at the same time, the interference between the alleles is avoided, thereby achieving efficient improvement of comprehensive traits such as crop yield, quality and resistance. Molecular marker-assisted selection breeding has the advantages that the identification of marker genotypes can be performed at any stage of low generation and plant growth, codominant molecular markers allow identification of recessive genes at the heterozygous stage, and the selection of the gene of interest is not affected by gene expression and environmental conditions. Molecular marker-assisted selection breeding is a means of applying molecular markers to crop improvement. The basic principle is to use the molecular markers closely linked to the target gene or to express co-segregation to target individuals and to screen the whole genome, thereby reducing the linkage and cumbersome, obtaining the desired individuals, and achieving the purpose of improving breeding efficiency.

MAS is based on different molecular markers, such as SSR markers, SNP markers, CAPS markers, etc., but the principles and steps are basically the same, although there are differences in the operation methods, there are a large number of related articles and books in the field. It has become a very common technique in the field of breeding and is well known to those skilled in the art. The basic steps include DNA extraction, PCR-labeled amplification, gel electrophoresis, and/or results (band-type) analysis.

The present invention refers to the DNA extraction method of Temnykh et al. (2000), and extracts genomic DNA for each representative plant of each strain.

The SSR markers of the perennial genetic loci are closely linked to the polymorphic SSR markers, and the single-strand genomic DNA is used as a template for polymerase chain reaction (PCR).

The products of the PCR reaction are separated by 8% non-denaturing polyacrylamide gel electrophoresis. After silver staining, the bands of the bands are discriminated and recorded with reference to the amplification bands of the parents, and the target genotypes are screened.

In the present invention, a perennial rice line is used as a donor, and in a preferred embodiment, the second F₁ generation is obtained by using the PR23 and/or PR24 perennial rice line as a donor and the second cultivated rice as a receptor. In a preferred embodiment, the second F₁ generation is F₁ (Chujing 28/PR24). The second F₁ generation is backcrossed and/or self-crossed with the second cultivated rice female parent, and each generation is traced and identified by closely linked molecular markers of the corresponding genetic loci (SSR marker, Table I), and the perennial rice carrying the perennial genetic locus of Oryza longistaminata is obtained by screening. Here, the purpose of the backcrossing is to clear the genetic background, using the MAS to leave the desired trait, and the others are consistent with the recurrent parent. Therefore, in the present invention, the sequence and the number of repetitions of the backcrossing and/or self-crossing of the second F₁ generation and the second cultivated rice are not particularly limited, as long as the perennial rice carrying the perennial genetic locus of Oryza longistaminata can be finally screened out. Wherein, in a preferred embodiment, the backcrossing is performed once, or is performed continuously twice, three times, four times or more; in a preferred embodiment, self-crossing is performed once, or is performed continuously twice, three times, four times or more. In a preferred embodiment, a single or continuous backcross and a single or continuous self-crossing can be alternately carried out.

In the present invention, the tracking and identification of the genetic locus is a MAS process, thereby obtaining a single plant with a perennial (asexual reproductive property) genetic locus of Oryza longistaminata.

The present invention cultivates and obtains the desired perennial rice by introducing the genetic locus for controlling the asexual reproduction characteristic (perennial) in Oryza longistaminata in the annual cultivated rice in the field, and realizes that the perennial rice plant can grow continuously for two or more years at a time. The present invention can also utilize the combination of different perennial genetic loci to cultivate perennial rice adapted to different ecological types, and from the second season, the steps of re-seeding, breeding, transplanting, etc. are eliminated, which has reduced the production cost of rice production and improved the production efficiency of rice production.

The technical solutions in the embodiments of the present invention will be clearly and completely described below in conjunction with the examples of the present invention for the purposed of making the purpose, technical scheme and advantages of the embodiment of the invention clearer. All other embodiments obtained by a person of ordinary skill in the art based on the embodiments of the present invention without creative efforts are within the scope of the present invention.

### EXAMPLES

Experimental Materials and Methods

Experimental materials include the following:
Oryza longistaminata: collected from Niger and provided by Dr. Hiroshi Hyakutake of the Japan Institute of Physics and Chemistry;
Cultivated rice RD23: indica variety from Thailand;
Cultivated rice Chujing 28: cultivated by Yunnan Chuxiong Agricultural Science Institute, the elite variety in Yunnan Province.

Hybridization methods are widely known in the art and are well within the abilities of those skilled in the art, and may be specifically referred to as Crop Breeding—China Agricultural University Press.

MAS and molecular marker detection of perennial genetic loci referred to the DNA extraction method of Temnykh et al. (2000), and genomic DNA was extracted from each representative strain of each strain. Polymerase chain reaction (PCR) was performed on each genomic DNA as a template for polymorphic SSR markers closely linked by perennial genetic loci. The products of the PCR reaction were separated by 8% non-denaturing polyacrylamide gel electrophoresis. After silver staining, the bands of the bands were discriminated and recorded with reference to the amplification bands of the parents, and the target genotypes were screened.

The materials and methods involved in this case were conventional materials and methods unless otherwise stated.

### Example 1

### Cultivation of F₁(RD23/O. longistaminaia) Generation

After RD23 as the female parent was emasculated, Fi(RD23/O. longistaminata) plants were obtained from the young embryos after direct pollination by O. longistaminata as the male parent. In the flowering period, the anthers were not cracked, and had about 30% of the pollen fertility, and the rhizomes behaved between the male parent and the female parent.

### Example 2

### Cultivation of Perennial Rice Lines PR24 and PR12-1375

The F₁(RD23/O. longistaminata) generation obtained in Example 1 was planted, and F₂ seeds were obtained by forced self-pollination of F₁ plants, and these seeds were cultured in ¼ MS medium (3% sucrose+0.7% agar, pH value of 5.8) to obtain seedlings, and transplanted by post-emergence seeding, and finally the isolated F₂ plants were obtained for screening.

The isolated F₂ plants were screened using one or more major QTL loci selected from Rhz2 and Rhz3, and one or more minor QTL loci selected from QRl1, QRbd2, QRn2, QRn3, QRn5, QRn6, QRl6, QRn7, QRl7, and QRl10 to select a perennial rice line PR24 (Perennial Rice 24, PR24) having short rhizomes. Verified by molecular detection, the line carried the perennial genetic loci Rhz2(Chr3), Rhz3(Chr4), QRn2(Chr2), QRbd2(Chr2), QRn7(Chr7), QRn10(Chr10) from O. longistaminata (Table 2), and confirmed by rice production practice, its yield performance was stable, agronomic traits were excellent, and it had good perennial properties. A perennial rice line PR12-1375 with long rhizomes was screened and bred. Verified by molecular detection, the line carried the perennial genetic loci Rhz2(Chr3), Rhz3(Chr4), QRn3(Chr3), QRbd2(Chr2), QRl7(Chr7), QRn10(Chr10) from O. longistaminata (Table 2), which has a strong environmental adaptability while obtaining a certain yield, adapting to dryland planting, and at the same time, because the rhizomes can increase soil coverage, thereby reducing soil erosion and protecting the ecological environment.

Table 2 lists the genotypes of the lines used in the examples.

TABLE 2
List of genotypes

| | Perennial genetic locus (chromosome) | | | | | | | |
|---|---|---|---|---|---|---|---|---|
| | QRn2 | QRn3 | QRbd2 | Rhz2 | Rhz3 | QRn7 | QRn10 | QRl7 |
| | (Chr2) | (Chr3) | (Chr2) | (Chr3) | (Chr4) | (Chr7) | (Chr10) | (Chr7) |
| Line name | RM327 | RM282 | RM71 | OSR16 | RM119 | RM125 | RM271 | RM505 |
| RD23 | aa | bb | cc | dd | ee | ff | gg | hh |
| F₁(RD23/O. longistaminata) | Aa | Bb | Cc | Dd | Ee | Ff | Gg | Hh |
| O. longistaminata | AA | BB | CC | DD | EE | FF | GG | HH |
| PR24 | AA | bb | CC | DD | EE | FF | GG | hh |
| PR12-1375 | aa | BB | CC | DD | EE | ff | GG | HH |
| Chujing 28 | aa | bb | cc | dd | ee | ff | gg | hh |
| PR2428 | AA | bb | CC | DD | EE | FF | GG | hh |

Note:
A is the female parental band type, B is the male paternal band type, H is the heterozygous band type, Rhz represents the major QTL locus of the rhizome; Q represents the minor QTL locus (QTL); Rn represents the number of rhizomes per plant; Rbd: degree of rhizome branching; Rl: average length of rhizomes; numbers indicate on which chromosome the locus is located.

### Example 3

### Perennial Rice Genetic Improvement

The artificial emasculation was carried out with the second cultivated rice Chujing 28 as the female parent, and the perennial rice PR24 was used as the male parent to obtain the F₁(Chujing 28/PR24) generation.

F₁(Chujing 28/PR24) and Chujing 28 were backcrossed 4 times, followed by 4 times of self-crossing. Using SSR-based Molecular Marker-Assisted Selection (MAS) breeding techniques in the backcrossing and self-crossing process, the perennial loci Rhz2(Chr3), Rhz3(Chr4), QRn2(Chr2), QRbd2(Chr2), QRn7(Chr7), QRn10(Chr10) were detected, and plants carrying these loci were selected for further backcrossing and self-crossing, until homozygous stability, and a new perennial rice line was obtained, named Perennial Rice 2428, i.e. PR2428 (Table 2).

### Example 4

### Perennial Rice Production Test

In addition to its perennial nature, the perennial rice PR24, which carried perennial genetic loci Rhz2(Chr3), Rhz3(Chr4), QRn2(Chr2), QRhd2(Chr2), QRn7(Chr7), QRn10(Chr10), has excellent characteristics such as compact plant type, high and stable yield, and is suitable for production. Perennial rice PR2428, in addition to perenniality, also has the excellent characteristics of Chujing 28, such as compact plant type, strong resistance, and high and stable yield.

The perennial rice varieties PR24, PR2428 and RD23 (annual rice control) were planted in the same field in the Xishuangbanna experimental field. After four seasons of field trials in two years, the results are shown in the following table (Table 3):

TABLE 3
Perennial rice field test results

| | | First season | Second season | Third season | Fourth season |
|---|---|---|---|---|---|
| Planting | | production | production | production | production |
| area | Variety | (kg/mu(666.7 m²)) | (kg/mu(666.7 m²)) | (kg/mu(666.7 m²)) | (kg/mu(666.7 m²)) |
| Location | RD23 | 488 | 0 | 0 | 0 |
| 1 | PR24 | 490 | 462 | 500 | 475 |
| | PR2428 | 515 | 473 | 509 | 480 |
| Location | RD23 | 518 | 0 | 0 | 0 |
| 2 | PR24 | 521 | 517 | 532 | 508 |
| | PR2428 | 587 | 558 | 604 | 562 |

Note:
The first season was early rice of the first year, the second season was late rice of the first year, the third season was early rice of the second year, the fourth season was late rice of the second year; the planting conditions and density of the location 1: natural conditions, 7,000 plants/mu; the planting conditions and density of the location 2: natural conditions, 11,000 plants/mu.

It can be seen from the above table that perennial rice can be planted once, continuously harvested by asexual reproduction for 4 times in two years, and has perenniality; genetic modification through MAS technology can obtain more new varieties of perennial rice with wide adaptability, high yield and high quality.

### Example 5

### Perennial Rice Production Test

The perennial rice line PR12-1375, which carries perennial genetic loci Rhz2(Chr3), Rhz3(Chr4), QRn3(Chr3), ORbd2(Chr2), QRl7(Chr7), QRn10(Chr10), had long rhizomes and could be perennial. The line also had excellent characteristics such as strong drought resistance and strong disease resistance. Although the yield was relatively lower than that of common cultivated rice, PR12-1375 was suitable for dry/mountain production and can obtain a certain yield.

The perennial rice variety PR12-1375 and the annual land rice IRAT104 (control) were planted on the slopes of Lancang or Menglian hillside. After four seasons of production test in two years, the results showed that compared with IRAT104, PR12-1375 had a perennial, could obtain a certain yield in continuous multiple seasons, and could effectively prevent soil erosion (Table 4).

TABLE 4
Yields of perennial rice line PR12-1375 in mountain area

| | First season | Second season | Third season | Fourth season |
|---|---|---|---|---|
| | production | production | production | production |
| Variety | (kg/mu) | (kg/mu) | (kg/mu) | (kg/mu) |
| IRAT104 | 388 | 0 | 0 | 0 |
| PR12- | 230 | 212 | 222 | 203 |
| 1375 | | | | |

It can be seen from the above table that perennial rice can be planted once, continuously harvested by asexual reproduction for 4 times in two years, and has perenniality. Genetic modification through MAS technology can obtain new perennial rice varieties with strong drought resistance and strong disease resistance.

It should be understood that the above specific embodiments of the present invention are used only for illustrative illustration or explanation of the principles of the present invention and do not constitute a limitation of the present invention. Therefore, any modifications, equivalent substitutions, improvements, etc., which are made without departing from the spirit and scope of the invention, are intended to be included within the scope of the present invention. In addition, the claims attached to the present invention are intended to cover all variations and modifications falling within the scope and boundaries of the claims attached, or equivalent forms of such scope and boundaries.

1. A method for cultivating perennial rice using an asexual propagation characteristic of Oryza longistaminata, comprising the following steps:
(1) crossing Oryza longistaminata as the male parent and a first cultivated rice as the female parent to obtain a first F₁ generation;
(2) self-crossing the first F₁ generation to obtain a first F₂ generation;
(3) screening out a first F₂ generation carrying a perennial genetic locus, wherein the perennial genetic locus includes a major QTL locus and a minor QTL locus;
(4) continuously self-crossing the first F₂ generation carrying the perennial genetic locus to obtain a progeny material, from which a perennial rice line is selected and bred, and the perennial rice line carries the perennial genetic locus, that is, it has perennial trait.

2. The method for cultivating perennial rice using an asexual propagation characteristic of Oryza longistaminata according to claim 1, further comprising the steps of:
(5) introducing the perennial genetic locus into a second cultivated rice through hybridization to obtain a second F₁ generation using the perennial rice line with perennial trait selected by step (4) as a donor and the second cultivated rice as a female parent;
(6) backcrossing the second F generation and the second cultivated rice as a male parent, and then self-crossing, and screening out the perennial rice carrying the perennial genetic locus.

3. The method for cultivating perennial rice using an asexual propagation characteristic of Oryza longistaminata according to claim 1, wherein the first cultivated rice is RD23.

4. The method for cultivating perennial rice using an asexual propagation characteristic of Oryza longistaminata according to claim 1, wherein the second cultivated rice is Chujing 28.

5. The method for cultivating perennial rice using an asexual propagation characteristic of Oryza longistaminata according to claim 1, wherein the major QTL locus includes Rhz2 and Rhz3, and the minor QTL locus includes QRl1, QRbd2, QRn2, QRn3, QRn5, QRn6, QRl6, QRn7, QRl7 and QRl10.

6. The method for cultivating perennial rice using an asexual propagation characteristic of Oryza longistaminata according to claim 1, wherein the perennial rice having perennial trait carries one or more perennial genetic loci selected from the group consisting of Rhz2, Rhz3, QRl1, QRbd2, QRn2, QRn3, QRn5, QRn6, QRl6, QRn7, QRl7 and QRl10.

7. The method for cultivating perennial rice using an asexual propagation characteristic of Oryza longistaminata according to claim 1, wherein the progeny material obtained by continuous self-crossing of the F₂ generation includes one or more selected from the group consisting of F₃generation, F₄ generation, F₅ generation, F₆ generation, F₇ generation, F₈ generation, F₉ generation, F₁₀ generation, F₁₁ generation and/or F₁₂ generation.

8. The method for cultivating perennial rice using an asexual propagation characteristic of Oryza longistaminata according to claim 1, wherein the second F₁ generation is backcrossed four times with the second cultivated rice male parent, and then self-crossed for 4 generations.
